(19) United States
(12) Patent Application Publication
(10) Pub. No.: US 2008/0050800 A1
(43) Pub. Date: Feb. 28, 2008

(54) METHOD AND APPARATUS FOR A MULTI-SYSTEM BIOENERGY FACILITY

(76) Inventors: Trevor McKeeman, Manhattan, KS (US); Greg Karr, St. George, KS (US); Praveen Vadlani, Manhattan, KS (US)

Correspondence Address: SCHWEGMAN, LUNDBERG & WOESSNER, P.A., P.O. BOX 2938, MINNEAPOLIS, MN, 55402, US

(21) Appl. No.: 11/844,307

(22) Filed: Aug. 23, 2007

Related U.S. Application Data
(60) Provisional application No. 60/823,358, filed on Aug. 23, 2006.

(51) Int. Cl. B09B 3/00; C12M 1/107

(52) U.S. Cl. 435/262.5; 435/300.1

(57) ABSTRACT

An alternative energy generating apparatus is provided for generating electricity, the apparatus includes an electric generating apparatus, wherein the electric generating apparatus produces flue gasses, at least one anaerobic digester adapted to supply biogas to the electric generating apparatus and at least one bioreactor configured to receive a least a portion of the flue gasses from the electric generating apparatus.

### RELATED APPLICATIONS

This application claims the benefit of provisional application No. 60/823,358 filed Aug. 23, 2006, entitled “METHOD AND APPARATUS FOR A MULTI-SYSTEM BIOENERGY FACILITY”, the disclosure of which is hereby incorporated by reference.

### TECHNICAL FIELD

This document relates to energy generation apparatus, systems and methods and more particularly to energy generation using alternative energy generation apparatus, systems and methods.

### BACKGROUND

Alternative energy, in one aspect, is energy derived in part, by non-traditional elements and/or non-traditional commercial processes. Traditional sources of energy include oil and its derivatives, coal, natural gas and electricity developed from nuclear reactions. Recent market developments have made traditional sources of energy more expensive than in the past. As a result, new research into alternative forms of energy has developed and resulted in new products and processes that produce energy from renewable, biological sources such as animal waste, crops such as corn, sorgum and soybeans, and biological entities such as algae. Some products and processes have been integrated into mainstream energy commerce, however there is a need to bring more alternative energy products and methods into the mainstream energy markets to reduce an anticipated high cost dependence on traditional energy sources.

### SUMMARY

Various embodiments provide energy generation apparatus including steam powered electrical generation equipment, anaerobic digesters and bioreactors. The anaerobic digesters are configured to generate methane and supply the methane to an ethanol production facility as combustible fuel for generating steam. The bioreactors are configured to receive nutrient rich waste water from the anaerobic digesters and carbon dioxide rich flue gas from the steam ethanol production facility to facilitate growth of biomass material, such as algae. In one embodiment, the algae is used as a source of triglyceride for the production of bio-diesel fuel.

In a method embodiment, animal waste is digested in a anaerobic process to produce methane, The methane is burned to produce steam for the purpose of producing ethanol. Combustion gases from the production of the steam and nutrient rich waste water from the anaerobic process are provided for facilitating the growth of biomass material such as algae. The biomass material is separated in to oils and solids. The biomass solids are fed to livestock to produce animal waste and are burned to generate steam energy for the production of ethanol. The biomass oils are exposed to a transesterification reaction to produce bio-diesel.

This Summary is an overview of some of the teachings of the present application and is not intended to be an exclusive or exhaustive treatment of the present subject matter. Further details about the present subject matter are found in the detailed description and the appended claims. The scope of the present invention is defined by the appended claims and their equivalents.

### BRIEF DESCRIPTION OF DRAWINGS

FIG. 1 illustrates a multi-system bioenergy facility according to one embodiment of the present subject matter.

FIG. 2 illustrates a multi-system bioenergy facility according to one embodiment of the present subject matter.

FIG. 3 illustrates a multi-system bioenergy facility according to one embodiment of the present subject matter.

FIG. 4 illustrates a multi-system bioenergy facility according to one embodiment of the present subject matter.

FIG. 5 is a flowchart of a method for producing alternative energy according to one embodiment of the present subject matter.

FIG. 6 illustrates one embodiment of a multi-system bioenergy facility.

### DETAILED DESCRIPTION

The following detailed description of the present invention refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides combination of independent energy generation apparatus to form various integrated energy generation apparatus and methods utilizing various forms of alternative inputs and generating various forms of energy outputs. In various embodiments, the apparatus and methods improve the economics of individual energy production facilities by integrating them operationally and geographically.

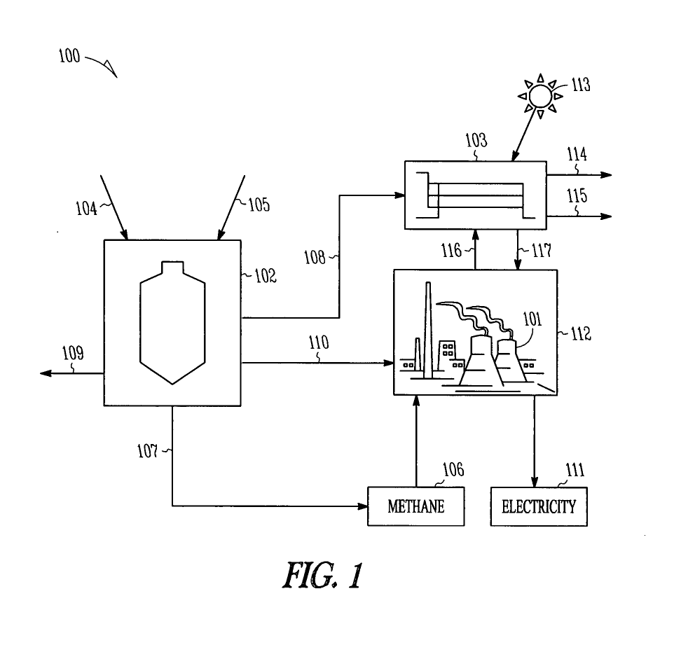

FIG. 1 illustrates a multi-system bioenergy facility 100 according to the present subject matter, including an electrical generating power plant 101, a anaerobic digester apparatus 102 and a bioreactor apparatus 103.

Anaerobic digestion is a multiphase biological process that involves sequential breakdown of complex organic waste using heterogeneous microorganisms. Anaerobic digestion includes both mesophilic and thermophelic digestion. The microbiology and biochemistry of anaerobic digestion involves several distinct pools of microbes, each performing specific task of the overall degradation. Typical anaerobic degradation process occurs in four main steps and involves at least three bacterial groups. The three bacterial groups are: the hydrolytic-fermentative bacteria (hydrolyzes the organic materials such as protein, carbohydrates and lipids, to amino acids, sugars, fatty acids and further to volatile fatty acids (VFA) along with hydrogen and carbon dioxide), acetogenic bacteria (converts the volatile fatty acids to acetic acid) and methanogenic bacteria (produce methane from acetic acid or from hydrogen and carbon dioxide).

In various embodiments, the anaerobic digesters 102 receive feedstock material such as wastewater 104, manure 105 or other waste stream with adequate Biological Oxygen Demand (BOD) and Chemical Oxygen Demand (COD) levels to produce biogas, including methane 106. Wastewater 104 and manure 105 are waste streams commonly associated with farms and other agricultural operations where livestock are raised, maintained and or processed. In various embodiments waste stream from a food processing plant, such as a cheese processing plant, or other food processing facility, supply the feedstock for the anaerobic digesters. In various embodiments, biogas 107, such as methane 106, from the anaerobic digesters 102 is used as a fuel to heat water and generate steam in the electric generating power plant 101. In addition to methane 106, the anaerobic digesters 102 provide a water waste stream 108 and a sludge 109. The waste water stream 108 of the anaerobic digesters 102 contains nitrogen, phosphates and potassium. In some embodiments, ammonia 110 is present in the waste water 108 of the anaerobic digesters 102. In various embodiments the ammonia is extracted made available for other uses, such as feedstock for an ammonia scrubber. In various embodiments, the sludge 109 produced by the anaerobic digesters 102 is used to seed subsequent anaerobic reactions. In various embodiments, the sludge 109 provides feedstock for the production of agricultural fertilizer as it can be processed to favorable nitrogen and phosphate content. Resultant sludge may also be used as livestock bedding.

The electric generating power plant 101 illustrated in the embodiment of FIG. 1 is a steam turbine based system adapted to use various fuels for producing the steam to rotate turbines. In various embodiments, steam production uses traditional fuels 112, such as coal and natural gas, as well as, alternative fuels, such as biogas 107, including methane 106. The steam is used to rotate turbines connected to an electric generator. The rotation of the generator produces electricity 111 for distribution on a power grid. In various embodiment, the generating power plant uses a gas turbine generator and burns biogas, including methane, directly to generate electricity.

The bioreactor 103 illustrated in FIG. 1 grows biomass material such as algae. The bioreactor process requires feedstock inputs including light 113, water, nutrients, CO2 and nitrogen. In various embodiments, the bioreactor 103 receives water through a fresh source, as well as, from the waste water stream 108 of the anaerobic digesters 102. In various embodiments, the waste water stream 108 of the anaerobic digesters 102 provides at least a portion of the nutrients and water required to facilitate the growth of biomass material in the bioreactor 103. The bioreactor 103 is designed to grow biomass in an efficient and timely manner. Harvested biomass provides various products such as biomass oil 114 and biomass solids 115. The value and composition of the biomass products will vary depending on the biomass grown in the bioreactor 103. For example, some algae-based biomass yield concentrations of more than 50% oil for a given weight of biomass. Biomass oil 114 is extracted from the biomass material, for example, by mechanical expression or solvent extraction. Biomass solids 115, such as algae solids, contain starches, proteins and cellulose materials. In various embodiments, biomass solids 115 are used as livestock feed and as fuel for combustion. In various embodiments, the bioreactor 103 is an open type system. An oval raceway pond, a round pond with a central mixing system, such as those commonly used for wastewater treatment or other open body of water are examples of open bioreactor systems. In various embodiments, the bioreactor is a closed system photobioreactor. A closed system photobioreactors are biorectors that require light to sustain biomass growth and which control exposure of the biomass to minimize contamination. Tube-type, greenhouse and trough type photobioreactors are examples of closed system photobioreactors. In various embodiments, the bioreactor apparatus 103 includes more than one bioreactor system.

In the embodiment of FIG. 1, biogas 107, such as methane 106, produced by anaerobic digesters 102 is used as a heating fuel to control the temperature of the anaerobic digesters 102. In some embodiments, where ambient temperatures can become cold, the temperatures of the anaerobic digesters are controlled to assure efficient operation. In various embodiments, a portion of the methane 106 produced by the digesters 102 is used as heating fuel for the temperature control of the digesters. In the embodiment of FIG. 1, the biogas from the anaerobic digesters 102 is used as a fuel to produce steam at the electrical generator 101. The heated steam is used to turn turbines connected to the rotor of an electrical generator. In various embodiments, the methane 106 is used as a co-fuel, along with traditional fuels 112, to generate the steam. Coal and natural gas are examples of traditional steam generator fuels 112. In various embodiments, the flue gas resulting from the production of steam contains carbon dioxide, CO₂. In the illustrated embodiment, the flue gas 116 is introduced to a biomass bioreactor 103. The CO₂ is dissolved in the nutrient rich water of the bioreactor and absorbed used by the growing biomass material. In various embodiments, waste water 108 from the anaerobic digesters 102 is introduced to the bioreactors 103 to provide nutrients such as nitrogen and phosphorous to facilitate the growth of the biomass material. In the illustrated embodiment, the anaerobic digester waste water 108 provides a portion of the nutrients required to facilitate biomass growth in the bioreactor. In the illustrated embodiment of FIG. 1, water 117 from the bioreactors is reused for cooling applications in the electric generator apparatus 101. In various embodiments, the bioreactor water 117 is treated to remove contaminants that may foul any downstream cooling equipment. Reuse of the bioreactor water diminishes the requirements for fresh water in a energy generation apparatus according to the present subject matter and provides a less negative overall environmental impact of the apparatus.

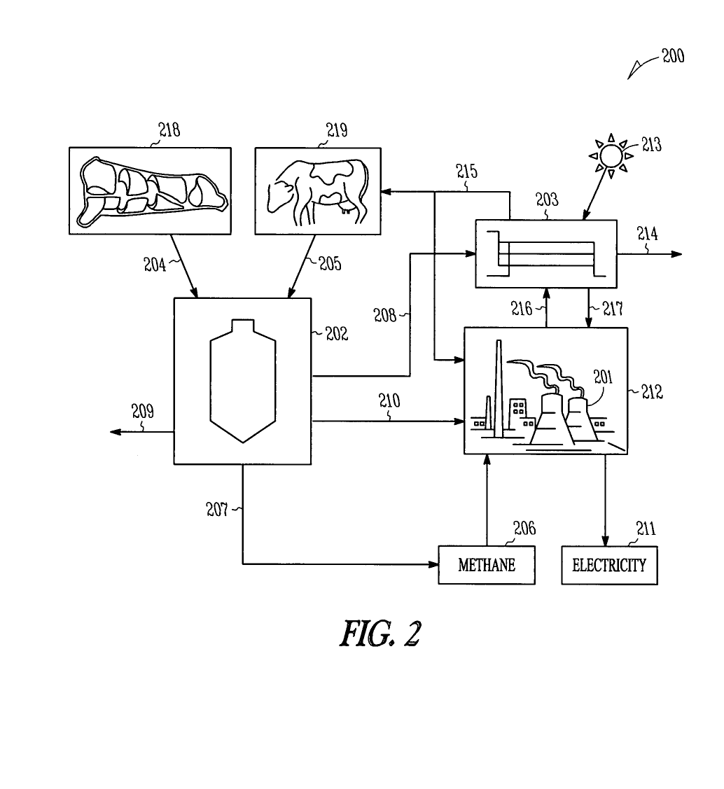

FIG. 2 illustrates a multi-system bioenergy facility 200 according to one embodiment of the present subject matter. FIG. 2 shows a alternative energy generation apparatus including an electrical generating power plant 201, at least one anaerobic digester 202 and a biomass bioreactor 203. The embodiment of FIG. 2 includes a livestock processing operation 218, such as a rendering plant, and a dairy operation 219. In various embodiments, the livestock processing operation 218 provides a waste stream 204 acceptable for use as a feedstock for the anaerobic digesters 202. Such a waste stream 204 includes the wastewater used for cleaning the livestock processing plant. The waste stream 204, after filtering coarse solids, includes blood, manure, hair, fat and bone fragments, among other things. In a traditional processing operations the waste stream would be treated and then released, for example, by spreading it over fields. In most cases, before the waste water can be released, it must be treated to reduce and dilute the biological load. However, the untreated, filtered waste provides an acceptable feedstock for the anaerobic digesters of the alternative energy generator embodiment of FIG. 2.

The multi-system bioenergy facility embodiment 200 of FIG. 2 shows a diary operation 219 providing feedstock to the anaerobic digesters 202. In addition to milk, a dairy operation produces manure 205. Manure is an acceptable feedstock to an anaerobic digester system for the production of biogas 207, including methane 206 and ammonia 210. The embodiment of the multi-system bioenergy facility of FIG. 2 shows a dairy operation 219 providing manure 205 as a feedstock to an anaerobic digester apparatus 202 for the production of biogas 207 including methane 206 and, in some cases, ammonia 210. In addition to biogas 207, the anaerobic digesters 202 provide a water waste stream 208 and a sludge 209. The waste water stream 208 of the anaerobic digesters 202 contains nitrogen, phosphates and potassium. In some embodiments, ammonia 210 may also be present in the waste water 208 of the anaerobic digesters 202. In various embodiments, the sludge 209 produced by the anaerobic digesters 202 is used to seed subsequent anaerobic reactions. In various embodiments, the sludge 209 provides feedstock for the production of agricultural fertilizer as it can be processed to favorable nitrogen and phosphate content.

The illustrated embodiment of FIG. 2 also shows the dairy operation 219 receiving biomass material 215 from the bioreactor apparatus 203. In various embodiments. the biomass material 215 provides a source of starches, proteins and cellulose materials suitable for feed of livestock such as dairy cows. The embodiment of FIG. 2 further shows the methane 206 generated by the anaerobic digesters 202 being used as a fuel for the electrical generator 201. In various embodiments, a portion of the methane 206 produced by the digesters 202 is used as heating fuel for the temperature control of the digesters. In the embodiment of FIG. 2, the biogas from the anaerobic digesters 202 is used as a fuel to produce steam at the electrical generator 201. The heated steam is used to turn turbines connected to the rotor of an electrical generator to produce electricity 211. In various embodiments, the methane 206 is used as a co-fuel, along with traditional fuels 212, to generate the steam. Coal and natural gas are examples of traditional steam generator fuels 212. In various embodiments, the flue gas 216, resulting from the production of steam, contains carbon dioxide, CO₂. In various embodiments, the biogas 207 is used as a heating fuel for temperature control of the anaerobic digesters 202. In various embodiments, ammonia 210 generated from the anaerobic digesters is used in the electrical generating power plant for air quality applications such as in flue gas scrubbers.

The embodiment of FIG. 2 shows flue gas 216 of the electrical generator being received by the bioreactor apparatus 203. In various embodiments, the flue gases 216 resulting from the combustion of coal to make steam for electrical generation, contains CO₂ and NOₓ that would normally be released into the atmosphere. In the embodiment of FIG. 2, captured flue gases 216 are exposed to the biomass material of the bioreactor 203. Portions of the CO₂ and NOₓ is absorbed by the biomass growing in the reactor and used by the biomass material, along with water, light 213 and other nutrients 208, to facilitate further growth of the biomass material. In various embodiments, waste water 217 exiting the bioreactor is used to generate steam in the electrical generating power plant 101. In various embodiments, oil 214 is extracted from the biomass material.

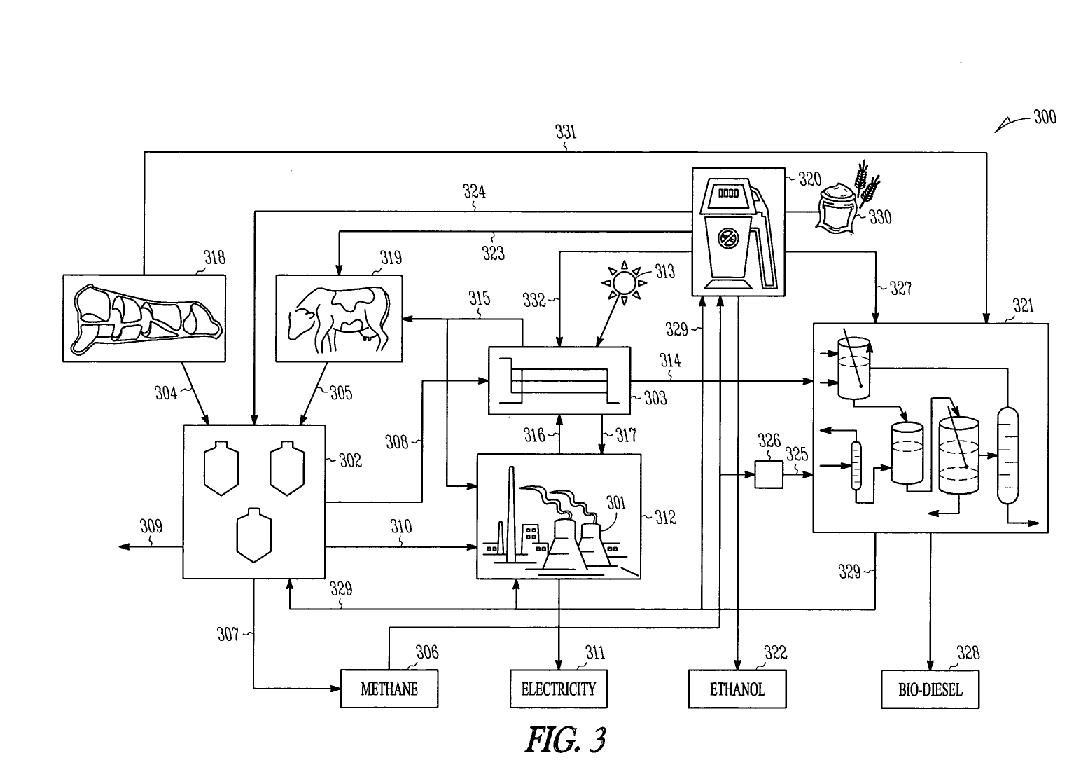

FIG. 3 shows one embodiment of the present subject matter where the multi-system bioenergy facility 300 includes an electric generation apparatus 301, a anaerobic digester apparatus 302, a bioreactor apparatus 303, a livestock processing apparatus 318, a dairy apparatus 319, a ethanol production facility 320 and a biodiesel production facility 321. The ethanol production facility 320 illustrated in the embodiment of FIG. 3 produces ethanol 322 using a yeast fermentation process of simple sugar substrates known in the art. The conversion of ethanol is conducted by the following chemical equation:

$$\begin{matrix} {C}_{6} {H}_{12} {O}_{6} + {H}_{2} O + Yeast ⇒ & 2 {CO}_{2} + 2 {C}_{2} {H}_{5} OH + {H}_{2} O \\ Glucose & Carbon Ethanol \\ & dioxide \\ 100 lbs & 49 lbs 51 lbs\end{matrix}$$

Feedstock for an ethanol production facility 320 according to the present subject matter include grains such as corn, sorghum and wheat 330. In general, a bushel of feedstock results in 1/3 becoming ethanol, 1/3 becoming distillers grain and 1/3 becoming CO₂. In various embodiments, CO₂ 332 is supplied from the ethanol production facility 320 to the bioreactor apparatus 303 to facilitate the growth of biomass material. In various embodiments, CO₂ 332 is supplied to the livestock processing facility for uses including, cold storage, as in dry ice packaging. Ethanol production requires approximately 24,00 BTUs of steam heat per gallon of ethanol, although requirements can vary substantially. In addition to heat and grain feedstock, the production of ethanol 322 also requires water. The multi-system bioenergy facility embodiment 300 of FIG. 3 shows the ethanol production facility 320 receiving grain feedstock 330 from traditional sources such as farms and cooperatives. The steam energy of the illustrated ethanol production facility is generated using energy resources produced or generated as by-products of other components of the multi-system bioenergy facility 300. In various embodiments, the steam energy fuel includes biomass solids 315 from the bioreactor 303, and biogas 307, including methane 306, produced by the anaerobic digesters 302, glycerol from the bio-diesel production facility, and excess steam and heat generated by the electrical generating plant. In various embodiments, biogas 307, including methane 306, are used as combustion fuel at the ethanol facility 320 to generate at least a portion of the required steam energy for ethanol production. In various embodiments, the water 317 exiting the bioreactors 303 is used to produce the steam of the ethanol production facility 320.

In various embodiments, residual product of the ethanol production facility 320, such as whole stillage, is used as feedstock for other components of the multi-system bioenergy facility 300. For example, the embodiment of FIG. 3 shows the wet distiller's grain 323 used as feedstock for the dairy operation and thin stillage 324 used as feedstock for the anaerobic digesters 302. Distillers grain is a component of whole stillage and includes insoluble, non-fermentable materials, high in fiber and protein. Thin stillage is another component of whole stillage and includes water soluble materials containing protein, unconverted starches and sugars. Untreated, distillers grain and thin stillage have a high water content making them expensive to transport long distances, therefore, embodiments of the present subject matter within close proximity of the ethanol facility, anaerobic digesters and bioreactors, result in significant economic advantages. Transportation cost can be reduced by drying the distillers grain and thin stillage. However, a drying operation will reduce the overall efficiency of the multi-system bioenergy facility.

The embodiment of FIG. 3 also includes a bio-diesel fuel production facility 321. In general, a bio-diesel fuel is a mixture of alkyl-esters with combustion and energy content properties similar to petroleum based diesel fuel. Bio-diesel fuel generally has a higher lubricity, flash point and cloud point values than petroleum based diesel fuel. Bio-diesel is generated by “cutting” triglycerides found in animal fats and vegetable oils by transesterification using simple alcohol in the presence of an alkali catalyst to produce alkylesters. Any simple alcohol may be used for the transesterification reaction, but methanol is typically used because of its low cost. Bio-diesel fuel can be used in standard diesel engines with little or no conversion.

The embodiment of FIG. 3 shows the triglyceride feedstock, such as tallow 331, for the bio-diesel production facility 321 coming from the livestock processing apparatus 318, In various embodiments, triglyceride feedstock is supplied to the bio-diesel production facility 321 from the bioreactor apparatus 303 and other outside sources. Tallow 331 is rendered from the suet or fat of bovine. In various embodiments, the bio-diesel production facility receives triglyceride feedstock from the bioreactors 303. In various embodiments, the biomass grown in the bioreactors can yield biomass with oil content greater than 50%. The biomass oil provides a source of triglyceride feedstock used in the production of bio-diesel fuel.

The embodiment of FIG. 3 shows the bio-diesel production facility 321 receiving methanol 325 for use in the transesterification reaction. Although the ethanol 322 produced by the alternative energy generation apparatus 300 may be used for the transesterification reaction, in general, most facilities would currently opt to use methanol as methanol is currently more economical in view of ethanol's superior commercial value. In the illustrated embodiment, methanol 325, converted 326 from the methane 306 generated at the anaerobic digesters, is used in the transesterification reaction. In various embodiments, an external source of methanol is used to supply the methanol.

The embodiment of FIG. 3, shows the bio-diesel fuel production facility 321 receiving vegetable oil 327 from the ethanol production facility 320. In various embodiments, vegetable oil 327, such as corn oil, can be extracted from the distillers grain of the ethanol production apparatus prior to distributing the distillers grain for feed. The extracted vegetable oils 327 provide an acceptable triglyceride source for the production of bio-diesel fuel 328. Other oils, such as canola oil, lard, cottonseed oil, palm oil, peanut oil, soybean oil, sunflower oil and waste vegetable oil from restaurants, for example, also provide acceptable sources of triglycerides for bio-diesel fuel production. However, some source oils will need to under go an acid pretreatment to convert the oil's Free Fatty Acid (FFA) content to methyl esters.

The multi-system bioenergy facility embodiment 300 of FIG. 3 also shows the bio-diesel production facility 321 receiving biomass oil 314 from the bioreactor apparatus 303. In various embodiments, the bioreactor apparatus 303 includes more than one bioreactor. In various embodiments, the bioreactors grow algae with oil content exceeding 50% of the dry weight of the algae. In various embodiments, algae oil from the bioreactor apparatus 303 is used to produce bio-diesel 328.

The multi-system bioenergy facility embodiment 300 of FIG. 3 shows the anaerobic digester apparatus 302 and the ethanol production facility 320 receiving glycerol 329 from the bio-diesel production facility 321. Glycerol 329 is a by-product of the production of bio-diesel fuel 328. In various embodiments, the resultant glycerol 329 is received by the anaerobic digester apparatus 302 for conversion to biogas 307, including conversion to methane 306. In various embodiments, glycerol 329 is supplied from the bio-diesel production facility as feed ration to the dairy operation 319 and/or the livestock processing facility 318. In various embodiments, the ethanol production facility 320 receives glycerol 329 for fuel in generating the steam energy necessary for ethanol production. In various embodiments, the bio-diesel production facility uses glycerol 329 as a fuel to provide the heat required to heat flash boilers used in the production of bio-diesel fuel 328.

In various embodiments, the anaerobic digester apparatus 302 includes more than one digester. The digesters receive feedstock such as waste water 304 and manure 305 from sources such as a livestock processing operations 318 and a dairy operations 319. The anaerobic digester apparatus produces biogas 307, sludge 309 and waste water 308. In various embodiments, CO₂ produced by anaerobic digestion is supplied to the bioreactor apparatus 303 to facilitate growth of biomass materials. In various embodiments, the biogas 307 contains methane 306. Waste water 308 from the anaerobic digesters 302 provide a source of nutrients, such as, carbon dioxide and nitrogen, that are used to facilitate growth of biomass in the bioreactors 303. In various embodiments, the waste water 308 of the anaerobic digesters contains ammonia 310. In various embodiments the waste water 308 from the anaerobic digesters is used to for cooling or steam generation applications in the electric generating power plant 301. In various embodiments, the biogas 307 produced at the anaerobic digester apparatus 302 is used as combustible fuel to produce steam at an alcohol production facility, such as an ethanol production facility 320. In the illustrated embodiment of FIG. 3, methane 306 generated at the anaerobic digester apparatus 302 is used as fuel to produce steam at the electric generating power plant 301. In various embodiments, the electrical generating steam is produced using traditional fuels 312, such as coal and natural gas. The steam is used to rotate turbines connected to the rotor of a generator. The generator produces electricity 311 for distribution on a power grid. In various embodiments, excess steam and thermal energy from the electrical generating power plant is supplied to the ethanol production facility 320. In various embodiments, excess steam and thermal energy from the electrical generating power plant is supplied to the bio-diesel production facility 321. In various embodiments, the bioreactor apparatus 303, receives nutrient rich waste water 308, sunlight 313 and flue gases 316 from the electrical generating power plant to facilitate the growth of the biomass in the bioreactor apparatus 303. In various embodiments, waste water 317 from the bioreactor apparatus is used for cooling applications in the electrical generating power plant 301. In various embodiments, some treatment of the bioreactor waste water may be required to protect the integrity of the cooling equipment.

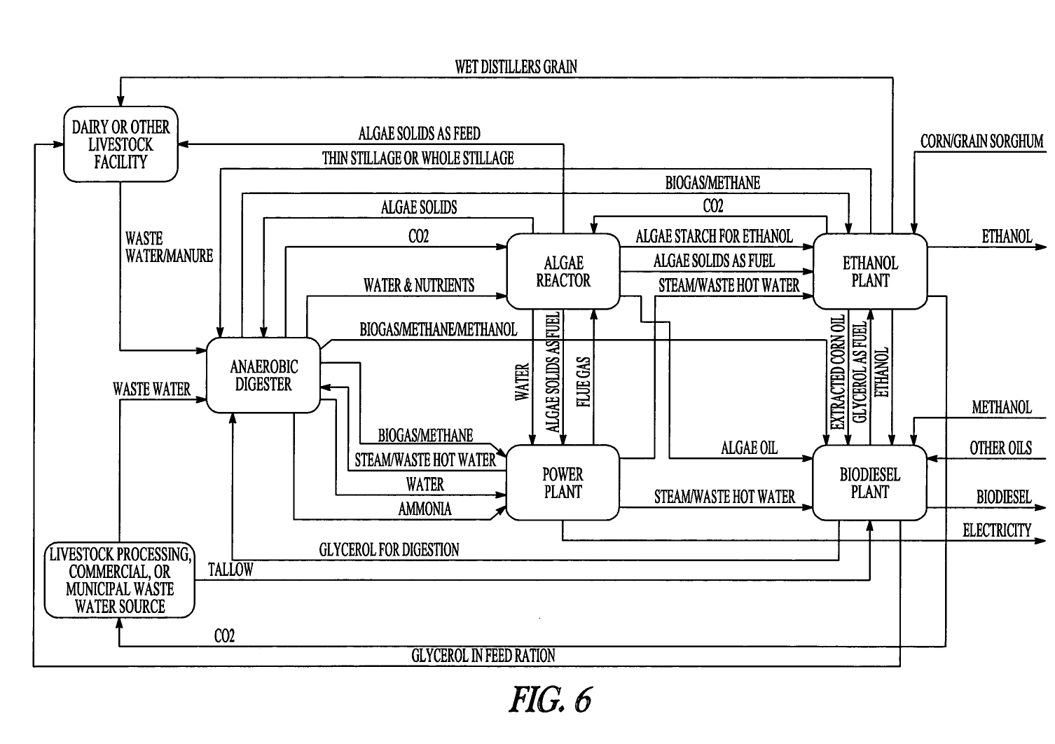

FIG. 6 illustrates one embodiment of a multi-system bioenergy facility. The illustrated embodiment includes many flows. Those of ordinary skill in the art will appreciate upon reading and comprehending this document that other embodiments my use fewer flows then illustrated. The description that follows describes the embodiment of FIG. 6.

The illustrated embodiment includes a dairy or livestock facility, a livestock processing or waste water facility, a anaerobic digester apparatus, an algae bioreactor apparatus, a power plant for producing electricity, an ethanol production facility and a bio-diesel production facility.

The dairy or livestock facility receives wet distillers grain from the ethanol facility, glycerol from the bio-diesel facility and algae solids from the bioreactor apparatus as nutritional supplements for the livestock or dairy cows. The dairy or livestock facility provides waste water and manure for feedstock materials to the anaerobic digester apparatus.

The livestock or food processing facility provides waste water to the anaerobic digester apparatus for digestion into biogas. The livestock or food processing facility supplies tallow for feedstock to the bio-diesel facility. The livestock or food processing facility receives CO₂ for cold storage applications from the ethanol facility.

The anaerobic digester apparatus provides waste water, biogas, ammonia and CO₂ to other systems of the multi-system bioenergy facility. Waste water from the anaerobic digester apparatus is used as a nutrient water stream by the algae bioreactor apparatus. Waste water from the anaerobic digester apparatus is used by the power plant for cooling application or steam production. Ammonia from the anaerobic digester apparatus is used by the power plant for scrubbing the flue gasses. Biogas, including methane, is used by the power plant, ethanol facility and the bio-diesel plant as a combustible fuel for thermal energy in various embodiments. Biogas, in the form of methane is converted to methanol to facilitate the production of bio-diesel in the bio-diesel plant. The anaerobic digester apparatus receives thin or whole stillage from the ethanol plant, waste water and manure from the dairy, livestock, food processing and waste water facilities, algae solids from the bioreactor apparatus and glycerol from the bio-diesel plant as feedstock for digestion.

The bioreactor apparatus receives nutrient from the waste water of the anaerobic digester apparatus, light from the sun, CO₂ from several sources and fresh water to facilitate the growth of algae and other biomass materials. Sources of CO₂ for the bioreactor include the ethanol plant, the anaerobic digester apparatus and flue gas from the power plant. Algae harvested from the bioreactor apparatus is used as feedstock for several systems of the multi-system bioenergy facility. Algea solids are supplied as feed for livestock and dairy operations. In various embodiments, algae solids are used for thermal energy in the power, ethanol and bio-diesel plants. Algae solids are a source of starch for production of ethanol. Algae solids are used as feedstock for the anaerobic digestor apparatus. In various embodiments, algae oil is extracted from the algae and used for the production of bio-diesel fuel. Waste water from the bioreactor apparatus is used for cooling and steam applications in the power plant.

The power plant produces electricity for distribution on a power grid, flue gas for use by the algae bioreactor apparatus and thermal and steam energy for use by the ethanol and bio-diesel plants.

In addition to the inputs mentioned above, the ethanol plant receives corn and grain sorgum for the production of ethanol. Glycerol from the biodiesel plant is used as a combustible fuel in the ethanol plant. In various embodiments, the ethanol plant supplies extracted vegetable oils, such as corn oil, to the bio-diesel plant for the production of bio-diesel. The ethanol plant supplies ethanol to the bio-diesel plant as either a combustible fuel or as a feedstock for conversion into methanol to facilitate the production of bio-diesel.

The bio-diesel plant receives oils from an external source, such as corn oil, canola oil, lard, cottonseed oil, palm oil, peanut oil, soybean oil, sunflower oil and waste vegetable oil, for the production of bio-diesel fuel. The bio-diesel plant receives methanol from an external source for the production of bio-diesel fuel.

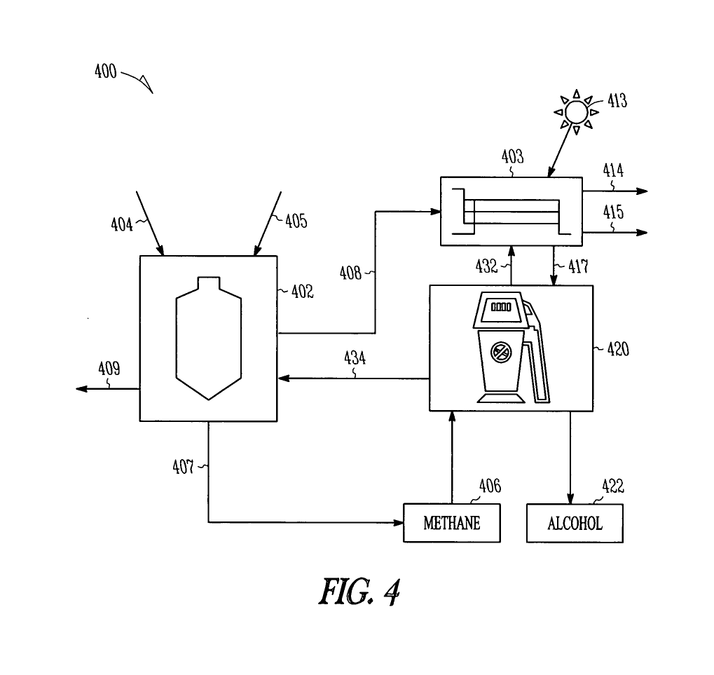

FIG. 4 illustrates one embodiment of a multi-system bioenergy facility 400 including an anaerobic digester apparatus 402, a bioreactor apparatus 403 and an alcohol production facility 420. In various embodiments, the anaerobic digester apparatus 402 receives feedstock materials such as manure 405 and waste water 404 from food or animal processing facilities. In various embodiments, municipal waste material forms at least a portion of the anaerobic digester apparatus feedstock. The anaerobic digester apparatus 402 digests the feedstock into components including biogas 407, sludge 409 and waste water 408. In various embodiments, biogas 407 is used as combustible fuel to produce steam in an alcohol production facility 420. In the illustrated embodiment of FIG. 4, methane 406 produced by the anaerobic digester apparatus 402 is used as combustible fuel for producing steam energy in the alcohol production facility 420. In the illustrated embodiment of FIG. 4, the anaerobic digester apparatus waste water 408 is used as a nutrient source for biomass material growing in the bioreactor apparatus 403. In various embodiments, the bioreactor apparatus 403 produces a continuous supply of biomass material, including algae. Continuous biomass growth is facilitated by exposing seed material to a stream of resources including light, nutrient rich water and CO₂. In the embodiment of FIG. 4, light is received from the sun 413, nutrient rich water 408 is received, in part, from the anaerobic digester apparatus 402 and CO₂ 432 is received from the alcohol production facility 420. In various embodiments, the alcohol production facility is an ethanol production facility. In various embodiments, the alcohol production facility is a butanol production facility or other alcohol production facility. In the embodiment of FIG. 4, bioreactor waste water 417 is used for producing steam in the alcohol production facility 420. As illustrated, in various embodiments, biomass, such as algae, is separated into solids 415 and oils 414. In various embodiments, the biomass solids 414, and distillers grain from the alcohol production facility, are used for livestock feed. In various embodiments, biomass solids 415 are supplied to the anaerobic digester apparatus 402 as feedstock for the production of biogas 407. In various embodiments, the biomass oil 414 is sold. In various embodiments, the biomass oil 414 is used as feedstock for the production of bio-diesel. In the illustrated embodiment of FIG. 4, thin stillage 424, resulting from the production of alcohol 422, is received by the anaerobic digester apparatus 402 to extract, by digestion, at least a portion of the energy content of the thin stillage in the form of biogas 407, such as methane 406. In various embodiments, whole stillage from the alcohol production facility is supplied to the anaerobic digesters as feedstock for the production of biogas.

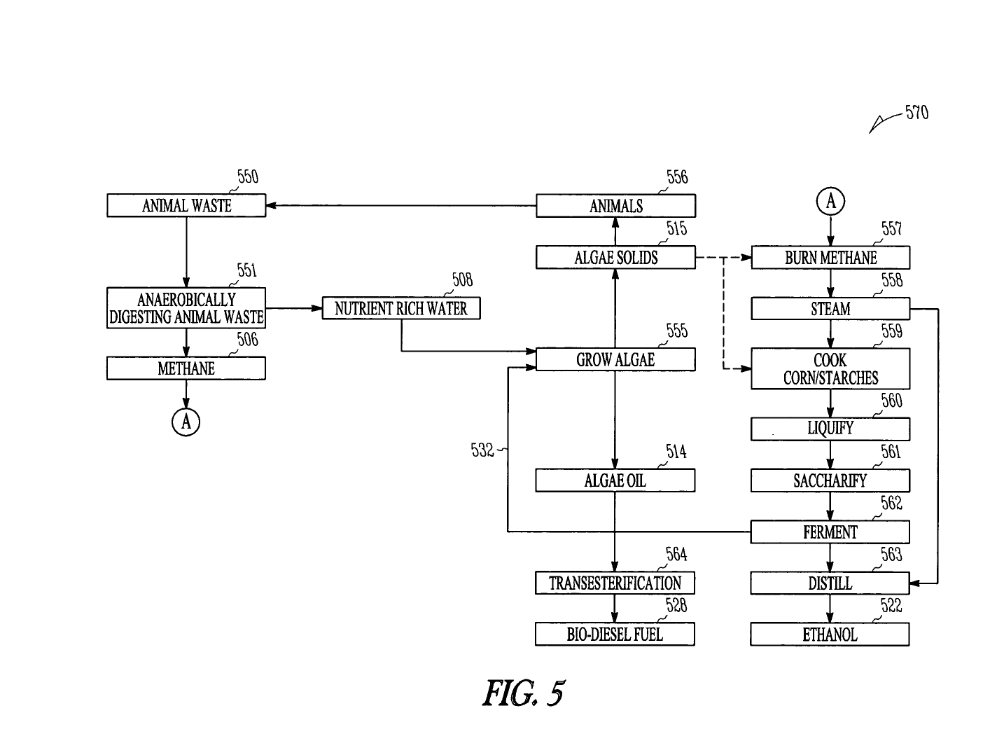

FIG. 5 is a method 570 of producing energy according to one embodiment of the present subject matter. The process begins by acquiring animal waste 550 and digesting the waste using an anaerobic process 551 to produce methane 506. The methane 506 is used to produce steam 558 for the production of ethanol 522. In various embodiments the methane may be burned with other fuels to produce the steam, such as coal, oil or natural gas. The ethanol is produced by cooking starch material 559, such as corn, with steam 558. Enzymes are then used to liquefy 560 and saccharify 561 the cooked starch. Fermentation 562 converts the sugars of the cooked starch to CO₂ 529 and ethanol and the ethanol 522 is extracted through distillation 563.

The CO₂ 532 produced by the ethanol production process is combined with nutrient rich waste water 508 from the anaerobic digestion process 551 to facilitate the growth of biomass material such as algae 555. The algae 555 is separated into algae oil 514 and algae solids 515.

In the illustrated embodiment, the algae solids 515 are fed to livestock animals 556, such as dairy cows, to produce animal waste 550. In various embodiments, the algae solids 515 are burned 557 as fuel to produce steam energy 558 for making ethanol 522. In various embodiments, the algae solids 515 provide a starch material 559 for the production of alcohol, such as ethanol or butanol.

In various embodiments, the algae oil 514 is combined with other triglyceride sources to produce bio-diesel 528. Other triglyceride sources include tallow and vegetable oil. Bio-diesel 528 is produced by subjecting the oils to a transesterification reaction 564 with a mixture of methanol and an alkali base. In various embodiments, the methanol is produced using the methane 506 generated by the prior anaerobic process 551. Sodium hydroxide and potassium hydroxide are examples of an alkali base material.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A multi-system bioenergy apparatus comprising:
an electric generating apparatus, wherein the electric generating apparatus produces flue gasses;
at least one anaerobic digester adapted to supply biogas to the electric generating apparatus; and
at least one bioreactor configured to receive flue gasses from the electric generating apparatus.

2. The apparatus of claim 1, further comprising at least one agricultural facility adapted to supply feedstock materials the at least one anaerobic digester.

3. The apparatus of claim 2, wherein the at least one agricultural facility includes at least one livestock processing operation configured to supply waste water to the at least one anaerobic digester.

4. The apparatus of claim 2, wherein the at least one agricultural facility includes at least one dairy operation configured to supply manure to the at least one anaerobic digester.

5. The apparatus of claim 1, further comprising an ethanol production facility configured to receive algae solids from the at least one bioreactor.

6. The apparatus of claim 1, further comprising a bio-diesel production facility configured to receive algae oil from the at least one bioreactor.

7. The apparatus of claim 1, wherein the at least one bioreactor is at least one closed system photobioreactor.

8. A multi-system bioenergy apparatus comprising:
an electric generating apparatus, wherein the electric generating apparatus produces flue gasses;
a bioreactor configured to receive flue gas from the electric generating apparatus;
an alcohol production facility configured to receive algae solids from the bioreactor;
an anaerobic digester configured to produce biogas for the alcohol production facility;
a livestock processing facility configured to supply waste water to the anaerobic digester;
a dairy facility configured to supply manure to the anaerobic digester; and
a bio-diesel production facility configured to receive algae oil from the bioreactor.

9. The apparatus of claim 8, wherein the anaerobic digester is configured to receive glycerol from the bio-diesel production facility.

10. The apparatus of claim 8, wherein the anaerobic digester is configured to receive thin stillage from the ethanol production facility.

11. The apparatus of claim 8, wherein the alcohol production facility is an ethanol production facility.

12. The apparatus of claim 11, wherein the ethanol plant is configured to supply wet distiller's grain to the livestock processing operation and the dairy facility for animal consumption.

13. The apparatus of claim 11, wherein the ethanol plant is configured to supply ethanol to the bio-diesel production facility.

14. The apparatus of claim 8 wherein the alcohol production facility is a butanol production facility.

15. The apparatus of claim 8, wherein the bioreactor is a closed system photobioreactor.

16. A method for producing energy comprising;
digesting agricultural waste using anaerobic digestion to produce a methane gas supply and a nutrient rich water supply;
burning the methane gas supply to produce steam;
growing algae using the nutrient rich water supply;
separating the algae into components including algae oil and algae solids;
producing bio-diesel fuel using at least a portion of the algae oil; and
producing ethanol using at least a portion of the steam;

17. The method of claim 16 further comprising feeding animals at least a portion of the algae solids.

18. The method of claim 16, wherein digesting animal waste includes digesting waste water from a livestock processing facility.

19. The method of claim 16, wherein digesting animal waste includes digesting manure from a diary operation.

20. The method of claim 16, wherein digesting animal waste includes digesting thin stillage from an ethanol production facility.

21. The method of claim 16, wherein producing ethanol using at least a portion of the steam includes:
providing a grain feedstock;
cooking the grain feedstock with the portion of the steam;
extracting an oil from the cooked grain feedstock; and
providing the oil as feedstock for producing bio-diesel.

22. The method of claim 21, wherein providing a grain feedstock includes providing a corn feedstock; and
extracting an oil from the cooked grain feedstock includes extracting corn oil from the cooked corn feedstock.

23. The method of claim 15, wherein producing bio-diesel fuel using at least a portion of the algae oil includes:
producing glycerol;
supplying the glycerol for combustion fuel; and
supplying the glycerol for anaerobic digestion into biogas.
